(19) United States
(12) Patent Application Publication
(10) Pub. No.: US 2006/0285636 A1
(43) Pub. Date: Dec. 21, 2006

(54) DENTAL IMAGE CHARTING SYSTEM AND METHOD

(75) Inventor: Michael R. Razzano, Marietta, GA (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: Interactive Diagnostic Imaging, Inc., Marietta, GA (US)

(21) Appl. No.: 11/468,128

(22) Filed: Aug. 29, 2006

Related U.S. Application Data
(63) Continuation of application No. 10/953,690, filed on Sep. 29, 2004.

(51) Int. Cl. A61B 6/14

(52) U.S. Cl. 378/38

(57) ABSTRACT

A computer system and method is disclosed that generates panoramic dental images and related dental charts. The system allows a user to specify which images the system should use for generating the panoramic image. The system then generates a panoramic image. An image-based chart and a graphic-based chart can be generated from the panoramic image. A user can input chart data on the charts using one or more input devices. A user can also select an option to have the system import chart data from an external source and display the data on the charts. Any inputted and imported data are displayed on the applicable chart for the user to view and analyze. A user can toggle between the individual images, the panoramic image, and the charts. A user can overlay one image or chart over the other. A user can also replace an individual image in the panoramic image.

### CROSS-REFERENCE TO RELATED APPLICATIONS

The present application is a continuation of prior application Ser. No. 10/953,690, filed Sep. 29, 2004, which is hereby incorporated by reference in its entirety.

### BACKGROUND

Dentists often do not have electronic access to the charting information they need in the operatory or lab environment itself. While dentists typically have access to a computer, the computer is typically located in an office outside of the operatory or lab and does not include software that is designed to work in an operatory or lab. These dentists may also have access to various paper-based sources within the operatory or lab, such as paper-based charts. However, these various paper-based sources are difficult to analyze together in a meaningful fashion. Furthermore, the paper-based sources then have to be later inputted into the computer system outside the operatory or lab for electronic analysis. There is a need for improved systems and methods for image charting so that dentists can have better access to resources that assist in analyzing data within environments such as operatories or labs.

### SUMMARY

One form of the present invention is a unique charting system.

Other forms include unique systems and methods to generate panoramic dental images and to create dental charts based on the panoramic dental images. Yet another form includes unique systems and methods to manage dental charts based on panoramic dental images.

Another form includes providing a computer system and method that generates panoramic dental images and related charts. The system allows a user to specify which images the system should use for generating the panoramic dental image. The system then generates a panoramic dental image. An image-based dental chart and a graphic-based dental chart can be generated from the panoramic dental image. A user can input chart data on one or both of the charts using one or more input devices. A user can also select an option to have the system import chart data from an external source and display the data on the chart. Any inputted and imported data are displayed on the applicable chart for the user to view and analyze. A user can toggle between the individual images, the panoramic image, and the charts. A user can overlay one image or chart over the other as desired. A user can also replace an individual image in the panoramic image as desired.

Yet other forms, embodiments, objects, advantages, benefits, features, and aspects of the present invention will become apparent from the detailed description and drawings contained herein.

### BRIEF DESCRIPTION OF THE DRAWINGS

FIG. 1 is a diagrammatic view of a computer system of one embodiment of the present invention.

FIG. 2A illustrates a first portion of a high-level process flow diagram for the system of FIG.1.

FIG. 2B illustrates a second portion of a high-level process flow diagram for the system of FIG. 1.

FIG. 3 is a process flow diagram for the system of FIG. 1 demonstrating the stages involved in generating a panoramic image.

FIG. 4 is a process flow diagram for the system of FIG. 1 demonstrating the stages involved in generating an image-based chart from a panoramic image.

FIG. 5 is a process flow diagram for the system of FIG. 1 demonstrating the stages involved in generating a graphic-based chart from a panoramic image.

FIG. 6 is a process flow diagram for the system of FIG. 1 demonstrating the stages involved in managing a panoramic image.

FIG. 7 is a simulated set of images that illustrate an example of individual images that can be used by the system of FIG. 1 for generating a panoramic image, as described in the procedures of FIGS. 2-3.

FIG. 8 is a visually simulated illustration of the programmatic process used by the system of FIG. 1 to align or wrap images to a panoramic plane, as described in the procedure of FIG. 3.

FIG. 9 is an illustration of a panoramic image generated by the system of FIG. 1, as described in the procedures of FIGS. 2-3.

FIG. 10 is an illustration of an image-based chart generated by the system of FIG. 1, as described in the procedures of FIGS. 2A-2B and FIG. 4.

FIG. 11 is an illustration of a graphic-based chart generated by the system of FIG. 1, as described in the procedures of FIGS. 2A-2B and FIG. 5.

### DETAILED DESCRIPTION

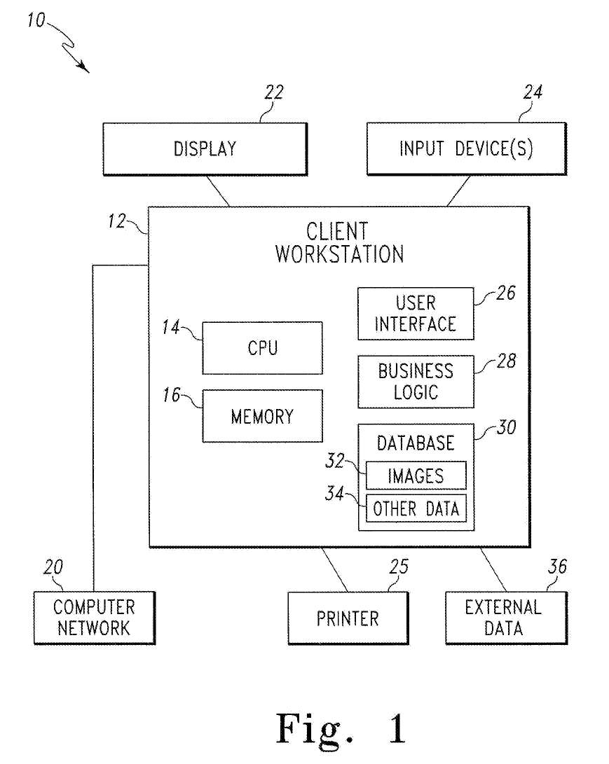

For the purposes of promoting an understanding of the principles of the invention, reference will now be made to the embodiment illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. FIG. 1 is a diagrammatic view of computer system 10 of one embodiment of the present invention. Computer system 10 includes client workstation 12. While client workstation 12 is illustrated as being a client computer, it should be understood client workstation 12 may be arranged to include both a client and server. Furthermore, it should be understood that while one computer is illustrated, more than one computer may be utilized in alternative embodiments.

Client workstation includes one or more processors or CPUs 14 and one or more types of memory 16. Each memory 16 includes a removable memory device, although not shown to preserve clarity. The processor may be comprised of one or more components configured as a single unit. Alternatively, when of a multi-component form, a processor may have one or more components located remotely relative to the others. One or more components of each processor may be of the electronic variety defining digital circuitry, analog circuitry, or both. In one embodiment, processor is of a conventional, integrated circuit microprocessor arrangement, such as one or more PENTIUM III or PENTIUM 4 processors supplied by INTEL Corporation of 2200 Mission College Boulevard, Santa Clara, Calif. 95052, USA.

Memory (removable or generic) is one form of computer-readable device. Memory may include one or more types of solid-state electronic memory, magnetic memory, or optical memory, just to name a few. By way of non-limiting example, memory may include solid-state electronic Random Access Memory (RAM), Sequentially Accessible Memory (SAM) (such as the First-In, First-Out (FIFO) variety or the Last-In-First-Out (LIFO) variety), Programmable Read Only Memory (PROM), Electronically Programmable Read Only Memory (EPROM), or Electrically Erasable Programmable Read Only Memory (EEPROM); an optical disc memory (such as a DVD or CD ROM); a magnetically encoded hard disc, floppy disc, tape, or cartridge media; or a combination of any of these memory types. Also, memory may be volatile, nonvolatile, or a hybrid combination of volatile and nonvolatile varieties.

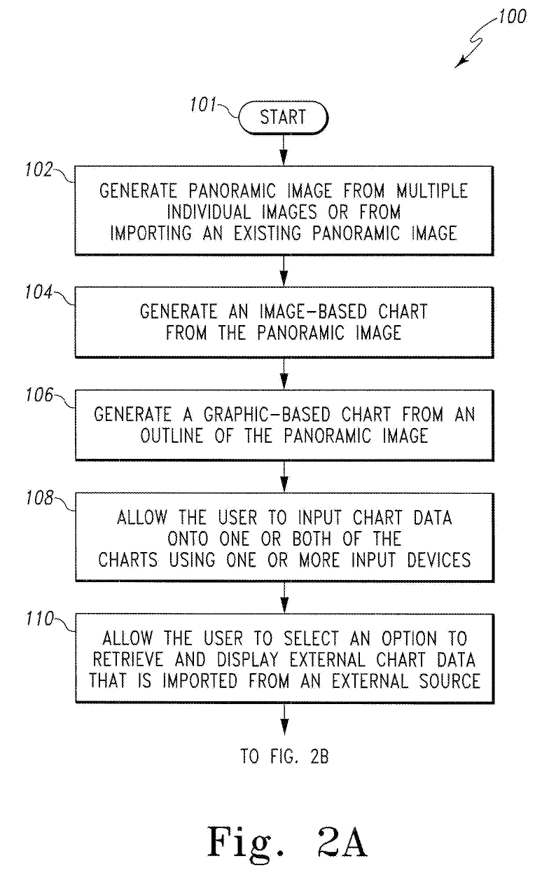

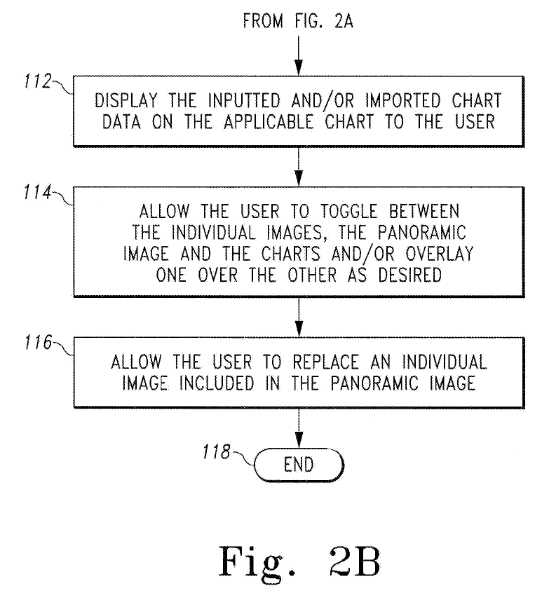

Client workstation 12 includes a display 22 and one or more input devices 24. Input devices 24 may include one or more operator input devices such as a keyboard, electronic pen input device, mouse, track ball, light pen, microtelecommunicator, gum pocket size measurement device, and/or a cavity detection device, to name just a few representative examples. Alternatively or additionally, client workstation 12 includes a printer 25. Alternatively or additionally, client workstation 12 has access to external data 36 in other systems and/or devices, such as external charting programs and digital cameras, to name a few non-limiting examples. Client workstation 12 also includes a user interface 26, business logic 28, and database 30. Database 30 stores image data 32 and other information 34 such as chart data. Business logic 28 includes the computer executed procedures described in FIGS. 2-6. Business logic 28 interfaces with user interface 26 to communicate with a user, and business logic 28 interfaces with database 30 to retrieve and store data. In one embodiment, client workstation 12 is disconnected from computer network 20. In another embodiment, client workstation 12 is connected to network 20.

Although only one client workstation 12 is shown to preserve clarity, more client workstations and/or servers could also be present. In such instances, multiple client workstations 12, displays 22, input devices 24, and printers 25 may be of the same respective type, or a heterogeneous combination of different computing devices. When more computers are present, client workstation 12 can be coupled to other computers over computer network 20. Computer network 20 could be in the form of a Local Area Network (LAN), Municipal Area Network (MAN), Wide Area Network (WAN), such as the Internet, a combination of these, or such other network arrangement as would occur to those skilled in the art. The one or more features provided by client workstation 12 can be provided on the same computer or varying other arrangements of computers at one or more physical locations and still be within the spirit of the invention. As one non-limiting example, a client-server arrangement can be used where the database 30 is located on one or more central servers that are accessible over computer network 20 by user interface 26 and business logic 28 that is present on one or more client computers 12. As another non-limiting example, a 3-tier/n-tier arrangement can be used over computer network 20 where the database 30 is located on one or more server computers, where at least some of the business logic 28 runs on one or more server computers, and where the user interface 26 resides on one or more client computers 12. Various other computer architecture arrangements are possible as is appreciated by one of ordinary skill in the computer art, and such arrangements are still within the spirit of the invention. The operating logic of system 10 can be embodied in signals in programming instructions, dedicated hardware, transmitted over computer network 20, or a combination of these.

In one embodiment, system 10 serves as an image charting system, with client workstation 12 containing a user interface 26, business logic 28, and database 30 that allow for creation and management of charts and related data. As one non-limiting example, system 10 can be used by a dentist to manage patient dental charts and associated information.

Referring additionally to FIGS. 2A-2B, one embodiment for implementation with system 10 is illustrated in flow chart form as procedure 100, which demonstrates a high level process flow diagram of some of the features provided by system 10. In one form, procedure 100 is at least partially implemented in the operating logic of system 10. Procedure 100 begins on FIG. 2A at start point 101 with generating a panoramic dental image from multiple individual images or from importing an existing panoramic image (stage 102). An image-based dental chart is generated from the panoramic image (stage 104). Alternatively or additionally to the image-based dental chart, a graphic-based dental chart is generated from an outline of the panoramic image (stage 106). A user can input chart data onto one or both of the charts using one or more input devices (stage 108). A few non-limiting examples of input devices include a mouse, keyboard, electronic pen, personal digital assistant (PDA), cavity detection device, and a gum pocket size measurement device. This is not an exhaustive list, and various other types of input devices can be used to input chart data onto one or both of the charts.

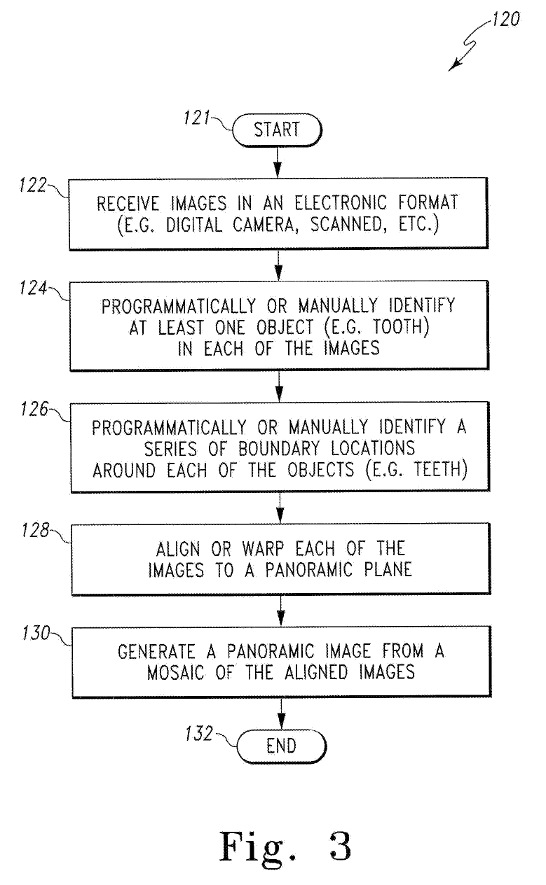

The user can select an option to retrieve and display external chart data that is imported from an external source (stage 110). Chart data can be imported from a variety of data sources and/or devices, such as an external program, a digital camera, or a personal digital assistant (PDA), to name a few non-limiting examples. Turning now to FIG. 2B, the inputted and/or imported chart data is displayed on the applicable chart to the user on display 22 (stage 112). The user can toggle between the individual images, the panoramic image and the charts and/or overlay one over the other as desired (stage 114). In one embodiment, the toggle feature provides the user with the ability to view the information in a variety of ways and to navigate among the charts and underlying images easily. There may be some instances where overlaying one image or chart on top of another image or chart is useful, such as for a dentist diagnosing a certain dental condition. If desired, the user can replace an individual image included in the panoramic image (116) with a new image. Upon replacement, a revised panoramic image can be generated in accordance with the procedure of FIG. 3. The process then ends at end point (118).

Turning now to FIG. 3, procedure 120 demonstrates the stages involved in generating a panoramic dental image (stage 102 in FIG. 2A). In one form, procedure 120 is at least partially implemented in the operating logic of system 10. Procedure 120 begins at start point 121 with receiving images in an electronic format (stage 122). Images that are received can include images that were captured with a digital camera, or that were scanned into an electronic format from a film-based image, to name a few non-limiting examples. The user can select an option in user interface 26 to specify from where the system can retrieve the images. At least one object (e.g. tooth) in each of the images is programmatically or manually identified (stage 124). In one embodiment, system 10 programmatically identifies the objects (e.g. teeth). Alternatively or additionally, the user has an option to identify at least some of the objects to assist system 10 in making a proper identification. A series of boundary locations around each of the objects (e.g. teeth) is programmatically or manually identified (stage 126). Each of the images is aligned or warped to a panoramic plane (stage 128). A panoramic image is generated from a mosaic of the aligned images (stage 130). The procedure then ends at end point 132.

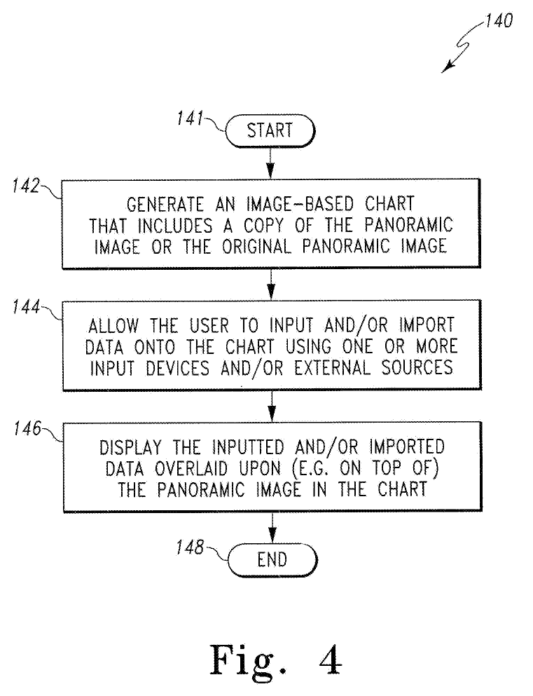

Turning now to FIG. 4, procedure 140 demonstrates the stages involved in generating an image-based dental chart from a panoramic image (stage 104 in FIG. 2A). In one form, procedure 140 is at least partially implemented in the operating logic of system 10. Procedure 140 begins at start point 141 with generating an image-based dental chart that includes a copy of the panoramic image or the original panoramic image (stage 142). The user can input and/or import data onto the chart using one or more input devices and/or external sources (stage 144). The inputted and/or imported data is displayed in an overlay fashion upon (e.g. on top of) the panoramic image in the chart (stage 146). By storing and/or displaying the chart data in an overlay fashion, the underlying image in the chart is preserved. Alternatively or additionally, the user can more easily work with the chart, such as by turning the display of chart data on and off as desired. Alternatively or additionally, the chart data can be permanently saved directly on the chart in non-overlay fashion. The procedure ends at end point 148.

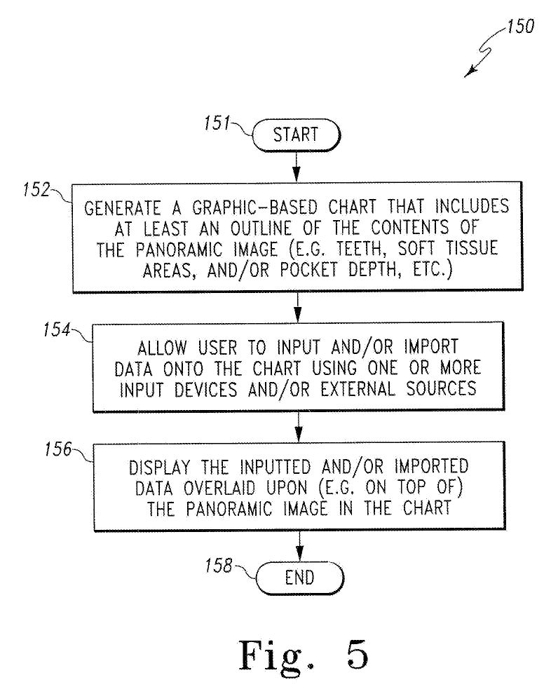

Turning now to FIG. 5, procedure 150 demonstrates the stages involved in generating a graphic-based dental chart from a panoramic dental image (stage 106 in FIG. 2A). In one form, procedure 150 is at least partially implemented in the operating logic of system 10. Procedure 150 begins at start point 151 with generating a graphic-based dental chart that includes at least an outline of the contents of the panoramic image (stage 152), such as an outline of the teeth, soft tissue areas, and/or pocket depth, to name a few non-limiting examples. The user can input and/or import data onto the chart using one or more input devices and/or external sources (stage 154). The inputted and/or imported data is displayed in an overlay fashion upon (e.g. on top of) the panoramic image in the chart (stage 156). Again, by storing and/or displaying the chart data in an overlay fashion, the underlying image in the chart is preserved. Alternatively or additionally, the user can more easily work with the chart, such as by turning the display of chart data on and off as desired. Alternatively or additionally, the chart data can be permanently saved directly on the chart in non-overlay fashion. The procedure ends at end point 158.

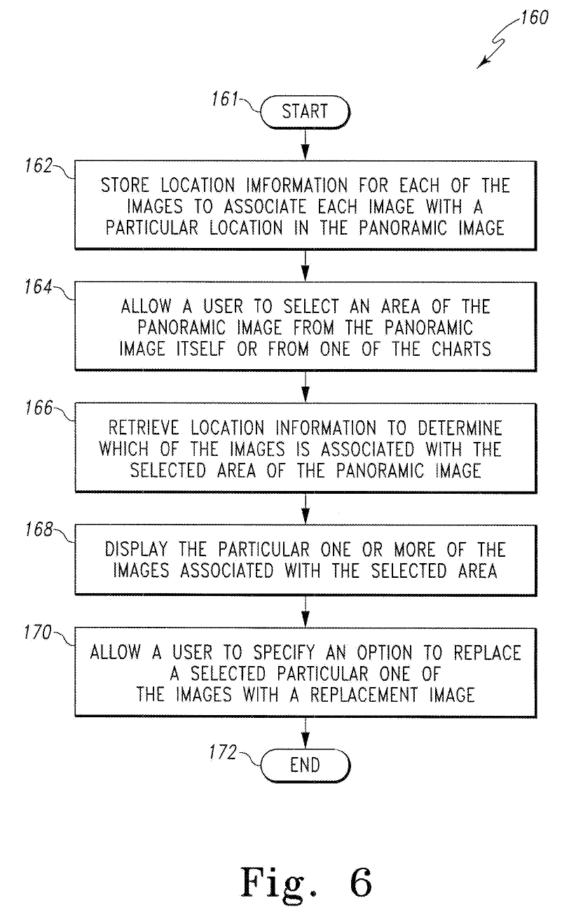

Turning now to FIG. 6, procedure 160 demonstrates the stages involved in managing a panoramic dental image (stage 116 in FIG. 2B). In one form, procedure 160 is at least partially implemented in the operating logic of system 10. Procedure 160 begins at start point 161 with storing in database 30 location information (other data 34) for each of the images to associate each image with a particular location in the panoramic image (stage 162). A user can select an area of the panoramic image from the panoramic image itself or from one of the charts (stage 164). Location information is then retrieved from database 30 to determine which of the images is associated with the selected area of the panoramic dental image (stage 166). The particular one or more of the images associated with the selected area is displayed on display 22 (stage 168). A user can specify an option to replace a selected particular one of the images with a replacement image (stage 170). Upon replacement, a revised panoramic image can be generated in accordance with the procedure of FIG. 3. The process then ends at end point 172.

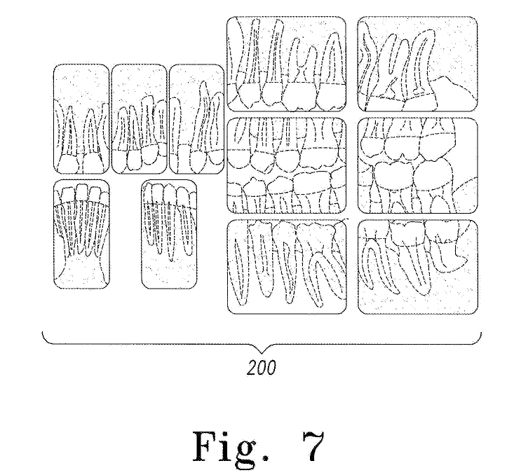

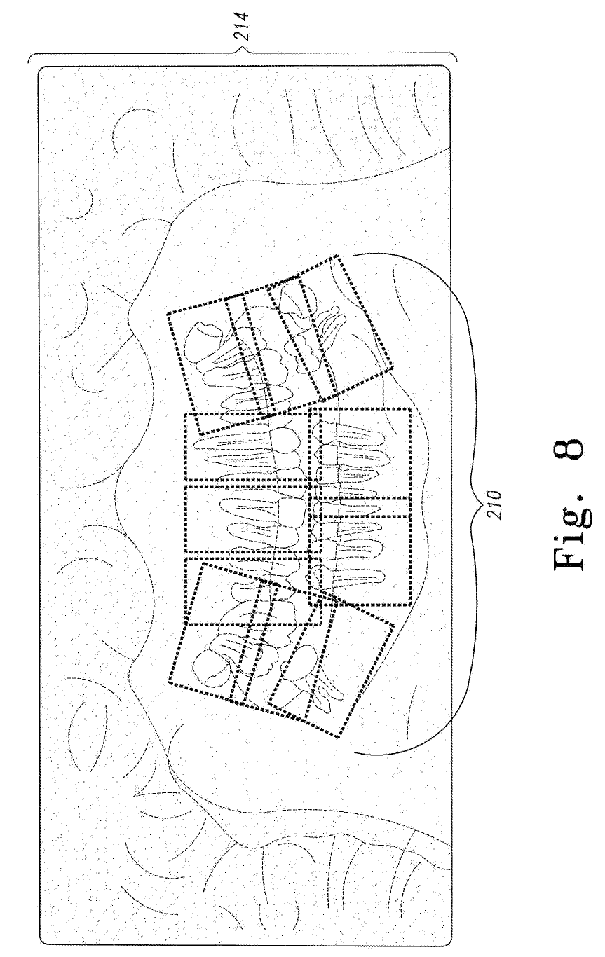

A hypothetical dental example will now be described in FIGS. 7-11 to illustrate the procedures of FIGS. 2-6. FIG. 7 illustrates a simulated set of dental images 200 used for generating a panoramic dental image, as described in FIGS. 2-3. Turning now to FIG. 8 (with continued reference to FIG. 7), a visually simulated illustration of the programmatic process used by system 10 in carrying out the procedures of FIGS. 2-3 is shown. Individual dental images 200 are received by system 10 in an electronic format (stage 122). Dental images 200 were captured with a digital camera, but film-based images that have been scanned to an electronic format, or other similar images types could also be used. In the current example, the dental images 200 are a full-mouth series of images, but more images or less images than a full-mouth series could be used. After the user selects an option to specify from where system 10 can retrieve the images, at least one object (e.g. tooth in this example) in each of the images is programmatically identified (stage 124) by business logic 28. Alternatively or additionally, the user has an option to identify at least some of the objects in images 200 to assist system 10 in making a proper identification.

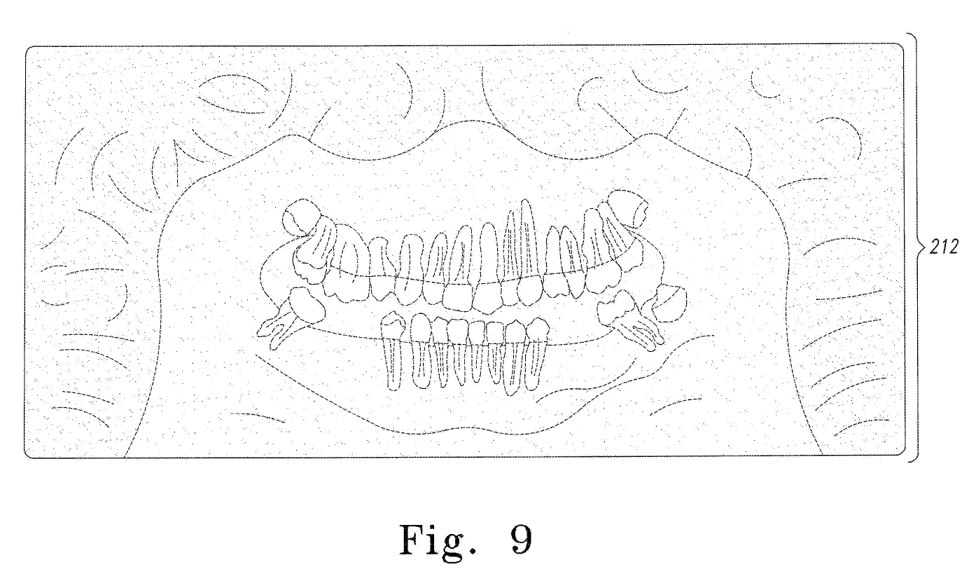

A series of boundary locations around each of the objects is programmatically identified (stage 126). Using the information obtained in the identification steps, system 10 has aligned or warped 210 each of images 200 to a panoramic plane (stage 128). Turning now to FIG. 9 with continued reference to FIG. 8, a panoramic dental image 212 is generated from a mosaic 214 of the aligned images 210 (stage 130).

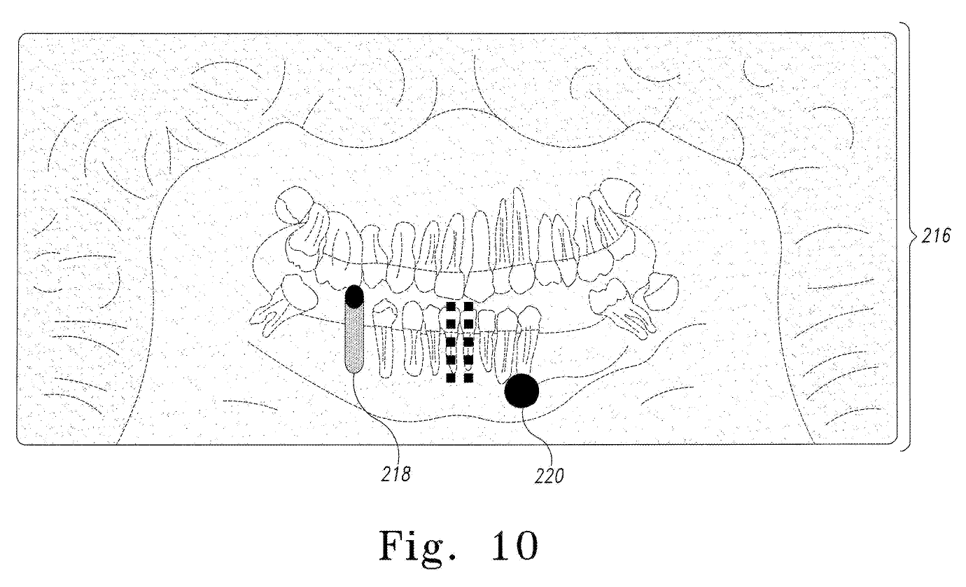

Turning now to FIG. 10, an illustration of an image-based dental chart 216 generated by system 10 in accordance with the procedures of FIGS. 2A-2B and FIG. 4 is shown. Image-based dental chart 216 includes a copy of panoramic dental image 212 (stage 142), and also includes symbols 218 and 220 (chart data) that were input by a user from an input device 24 (stage 144). The chart data (218 and 220) is displayed in an overlay fashion on top of the panoramic image included in chart 216 (stage 146). By overlaying the chart data (218 and 220), the underlying panoramic dental image is preserved. The user can more easily work with the chart, such as by turning the display of chart data on and off as desired.

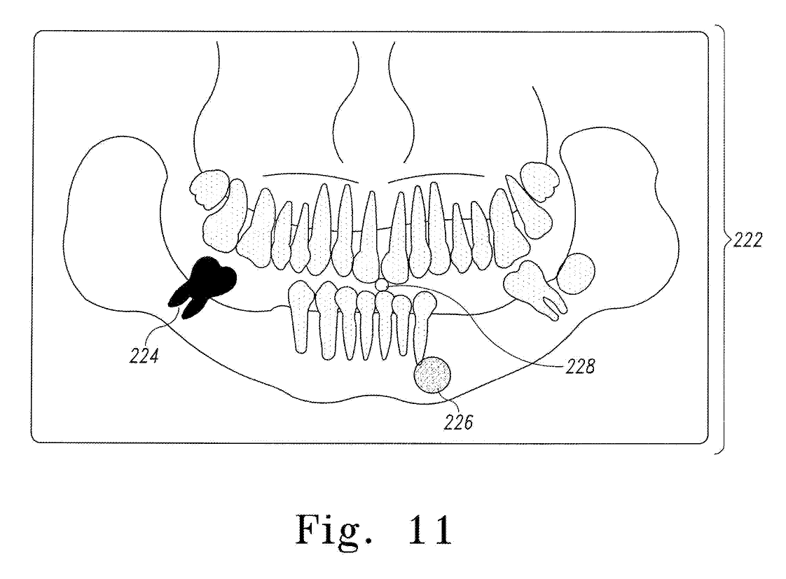

Turning now to FIG. 11, an illustration of graphic-based chart 222 generated by system 10 in accordance with the procedures of FIGS. 2A-2B and FIG. 5 is shown. In the current example of a panoramic dental image, graphic-based dental chart 222 includes an outline of the teeth, soft tissue areas, and pocket depth (stage 152). Graphic-based dental chart 222 includes symbols 224, 226, and 228 (chart data) that were input by a user from an input device 24 (stage 154). The chart data (224, 226, and 226) is displayed in an overlay fashion upon (e.g. on top of) the panoramic image in chart 222 (stage 156). Again, by storing and/or displaying the chart data in an overlay fashion, the underlying image in the chart is preserved. The user can more easily work with the chart, such as by turning the display of chart data on and off as desired.

In another embodiment of the present invention, a method is disclosed that comprises generating a panoramic dental image in an electronic format, said panoramic dental image illustrating a plurality of teeth; generating an image-based dental chart from the panoramic image; generating a graphic-based dental chart from the panoramic image, said graphic-based dental chart including a graphical outline of each of the teeth in the panoramic image; and wherein the image-based dental chart and the graphic-based dental chart are operable to receive and display data that is input using at least one of a plurality of input devices.

In yet another embodiment of the present invention, a method is disclosed that comprises receiving a plurality of dental images in an electronic format; identifying at least one tooth in each of the images; identifying a series of boundary locations around each tooth; aligning each of the dental images to a panoramic plane; generating a panoramic dental image from the aligned images; and generating at least one electronic dental chart that includes a chart image created from the panoramic dental image, said chart being operable to receive data that was input using at least one of a plurality of input devices, and said chart being further operable to display the data overlaid on top of the chart image.

In another embodiment of the present invention, a system is disclosed that comprises a business logic routine; a first computer coupled to the business logic routine, said first computer including at least one of a plurality of input devices, said second computer further including a display device; and wherein the business logic routine is operable to generate a panoramic dental image from a plurality of dental images, said panoramic dental image illustrating a plurality of teeth; to generate at least one electronic dental chart that includes a chart image created from the panoramic dental image; to display the at least one dental chart on the display to a user upon request; and to receive data input from the user from the at least one of the input devices, said data input including data to be displayed on the at least one dental chart.

In yet a further embodiment of the present invention, an apparatus is disclosed that comprises a device encoded with logic executable by one or more processors to generate a panoramic dental image in an electronic format from a plurality of dental images, said panoramic dental image illustrating a plurality of teeth; generate an image-based dental chart from the panoramic dental image; generate a graphic-based dental chart from the panoramic dental image, said graphic-based dental chart including a graphical outline of each of the teeth in the panoramic dental image; receive chart data from at least one of a plurality of input devices; and display the chart data overlaid on top of at least one of the dental charts.

All publications, prior applications, and other documents cited herein are hereby incorporated by reference in their entirety as if each had been individually incorporated by reference and fully set forth.

A person of ordinary skill in the computer software art will recognize that the client and/or server arrangements, user interface screen content, and data layouts could be organized differently to include fewer or additional options or features than as portrayed in the illustrations and still be within the spirit of the invention.

While the invention has been illustrated and described in detail in the drawings and foregoing description, the same is to be considered as illustrative and not restrictive in character, it being understood that only the preferred embodiment has been shown and described and that all equivalents, changes, and modifications that come within the spirit of the inventions as described herein and/or by the following claims are desired to be protected.

What is claimed is:

1. A method comprising:
receiving a panoramic dental image in an electronic format, said panoramic dental image illustrating a patient's teeth;
generating an image-based dental chart from the panoramic image;
generating a graphic-based dental chart from the panoramic image, said graphic-based dental chart including a graphical outline of each of the teeth in the panoramic image; and
wherein the image-based dental chart and the graphic-based dental chart are operable to retrieve and display external data that is imported from an external source.

2. The method of claim 1, wherein the external source is a dental charting program.

3. The method of claim 1, further comprising:
providing a feature that allows a user to toggle between the panoramic dental image, the image-based dental chart, and the graphic-based dental chart.

4. The method of claim 1, wherein said dental image reveals internal structures of said patient's teeth opaque to ordinary visible light.

5. The method of claim 1, wherein said panoramic dental image is a panoramic dental X-ray image.

6. The method of claim 1, wherein said dental image is a shadow picture made with X-rays.

7. The method of claim 1, wherein the image-based dental chart and the graphic-based dental chart are operable to receive and display data that is input using at least one of a plurality of input devices.

8. The method of claim 1, wherein the graphical outline further includes an outline of soft tissue areas and pocket depth.

9. The method of claim 7, wherein the data includes symbols.

10. The method of claim 7, wherein the at least one of the input devices is an electronic pen.

11. The method of claim 7, wherein the at least one of the input devices is a device that measures gum pocket size.

12. The method of claim 7, wherein the at least one of the input devices is a cavity detection device.

13. A system comprising:
a business logic routine;
a first computer coupled to the business logic routine, said first computer including at least one of a plurality of input devices, said first computer further including a display device;
wherein the business logic routine is operable to receive a panoramic dental image of a patient's teeth in an electronic format; to generate at least one electronic dental chart that includes a chart image created from the panoramic dental image; to display the at least one dental chart on the display to a user upon request; and to receive data input from the user from the at least one of the input devices; and to display said data input on the at least one dental chart.

14. The system of claim 13, wherein the business logic resides and executes on the first computer.

15. The system of claim 13, wherein the first computer is coupled to a second computer over a network, and wherein the business logic routine resides and executes on the second computer.

16. The system of claim 13, wherein said business logic routine is further operable to retrieve and display external data that is imported from an external source.

17. The system of claim 13, wherein the at least one of the input devices is an electronic pen.

18. The system of claim 13, wherein said at least one electronic dental chart is a graphic-based dental chart and wherein said chart image includes a graphical outline of each of the teeth in the panoramic dental image.

19. The system of claim 13, wherein said at least one electronic dental chart includes an image-based dental chart.

20. The system of claim 13, wherein said panoramic dental image is a panoramic dental X-ray image.

21. An apparatus, comprising: a device encoded with logic executable by one or more processors to:
receive a panoramic X-ray dental image in an electronic format, said panoramic dental image illustrating a plurality of teeth;
generate an image-based dental chart from the panoramic X-ray dental image;
generate a graphic-based dental chart from the panoramic X-ray dental image, said graphic-based dental chart including a graphical outline of each of the teeth in the panoramic dental image;
receive chart data from at least one of a plurality of input devices; and
display the chart data overlaid on top of at least one of the dental charts.

22. The apparatus of claim 21, wherein the device includes a removable memory device carrying a number of processor executable instructions to define the logic.

23. The apparatus of claim 22, wherein the removable memory device includes a disk.

24. The apparatus of claim 21, wherein the device is in the form of one or more parts of a computer network carrying one or more signals encoding the logic.

25. The apparatus of claim 21, wherein the device is operable to receive external data that is imported from an external source and display the external data overlaid on top of at least one of the dental charts.

26. The apparatus of claim 25, wherein the external source is a dental charting program.
